(19) United States
(12) Patent Application Publication
(10) Pub. No.: US 2026/0115030 A1
(43) Pub. Date: Apr. 30, 2026

(54) TRACTION ANKLE WRAP

(71) Applicant: Ronald M. Carn, Redding, CA (US)

(72) Inventor: Ronald M. Carn, Redding, CA (US)

(21) Appl. No.: 19/369,073

(22) Filed: Oct. 24, 2025

Related U.S. Application Data
(60) Provisional application No. 63/711,401, filed on Oct. 24, 2024.

(51) Int. Cl. A61F 5/01

(52) U.S. Cl. CPC A61F 5/0111

(57) ABSTRACT

The traction ankle wrap is of a unibody construction and may be made from a stretchable neoprene material. A rear support positionable behind a patient ankle, a base support positionable underneath a patient foot, and a series of straps extending therefrom are of a size and shape able to generally fold over and encompass at least part of the patient foot, the entire ankle, and/or at least a portion of the patient's lower leg, to provide support thereto, including anteriorly, when the patient is placed into traction with a traction splint.

### BACKGROUND OF THE INVENTION

The present invention generally relates a traction ankle wrap. More specifically, the present invention is directed to a traction ankle wrap for placing a relatively consistent or even force in and around the entire ankle, while simultaneously providing enhanced anterior support thereto, for use in connection with a traction splint.

Traction splints are medical devices that are used to stabilize and immobilize a limb, typically in cases of suspected fractures (e.g., the femur) or other serious injuries. Traction splints are generally designed to apply a pulling force along the length of the injured limb called “traction” to keep fractured ends of the bone in alignment to help reduce pain, prevent further injury, and facilitate safer transport. Some traction splints are designed for use in a hospital or other medical care facility, while other types of traction splints may be used in the “field” to help stabilize a patient injury for transport in an ambulance from one location to another, such as from the location of an accident to a hospital for definitive care.

Traction splints designed for use in the “field” are often relatively lightweight compared to traction splints designed for use in a hospital or other medical facility. As such, “field” taction splints are typically designed to be easily transported and applied to patients relatively quickly by first responders in outdoor or remote settings. In this respect, a field traction splint typically includes a relatively rigid frame or support structure (e.g., made from metal or durable plastic) that provides stabilizing support to the injured limb. A padded or cushioned leg support is typically designed to fit in and around the foot, ankle, and a lower part of the leg to provide further cushioned support and stability thereto. A set of straps located along the length of the padded or cushioned leg support are used to secure the injured limb within the padded or cushioned leg support to help ensure that the injured limb remains immobilized during transport. A traction mechanism, which may include devices like pulleys, ropes, or straps, applies a pulling force to the limb to apply traction through the lower extremity to stabilize and immobilize the fracture, particularly in long bones (e.g., the femur), by pulling on the ankle and transferring the pressure to the pelvis bone. Applying traction in this respect helps stabilize and align the femur bone and tends to reduce muscle spasms, thereby minimizing additional damage from sharp boney fragments. This also helps to alleviate pain and to prevent further injury when the patient is transported from the field to a medical facility (e.g., a hospital) in an emergency. The traction mechanism may include an adjustment feature so that the field traction splint can be used to fit the entire length of the injured limb, to achieve the desired traction.

There are also a number of “leg” traction devices known in the art that may be generally grouped together into a category of medical devices called “bucks” traction devices. Bucks traction devices are relatively large and bulky, and typically include complex assemblies. As such, bucks traction devices are typically only usable in a hospital setting due to their lack of portability and compatibility for use in the “field”. In this respect, bucks traction devices can be integrated as part of the care the patient receives in a hospital bed, including that bucks traction devices may be sterilized for use in an operating room (e.g., during orthopedic surgery). Some bucks traction devices, e.g., require attachment to a structure or frame such as a hospital bed for full functionality. As such, bucks traction devices are not suitable for use outside a care facility, such as in the field for use by emergency personnel.

In one prior art reference, U.S. Publication No. 2015/0366695 to Galloway, the contents of which are herein incorporated by reference in its entirety, discloses a limb stabilization device having soft bending actuators in around the joints, including the ankle. The Galloway ankle stabilizing feature provides assistive torques or continuous passive motion to joints by way of an elastic band extending from a top portion of the ankle to a lower foot portion thereof. As such, the Galloway ankle stabilizer only distributes compressive pressures across a relatively small area of the upper ankle and lower foot and is otherwise not designed to prevent the ankle from moving, which is undesirable for traction. To this end, the Galloway limb stabilizing device does not place a relatively consistent or even force in and around the entire ankle while also providing enhanced anterior support thereto.

In another example, U.S. Pat. No. 11,051,967 to Kramer, the contents of which are herein incorporated by reference in its entirety, discloses a process for treating femur fractures in children utilizing a functional bracing technique via an orthosis that provides compression over the fracture. The Kramer orthosis encloses around a fractured leg with a foot section and a strap that extends over the instep of the foot. Again, the foot section and strap only apply direct pressure to small areas on the foot, which is undesirable for traction because there are no forces in and around the entire ankle. Moreover, the orthosis has a leg section, upper leg section, and torso section that attach along the length of the patient. This is also undesirable because the device is complex and bulky, and impractical for quick and easy use in emergency settings, especially in the “field”.

Additionally, U.S. Pat. No. 8,845,569 to Buckman, the contents of which are also incorporated by reference in its entirety, discloses an adjustable traction splint designed to provide better control and stabilization of bone fractures in mammals. The Buckman design includes an inflatable member that expands and locks into position in and around the leg. The traction device otherwise does not provide distributed compressive support in and around the entirety of the foot, ankle, and/or calf. The splint has an integral backboard or stretcher to stabilize an appendage, but Buckman does not disclose the use of an ankle harness, boot, or specific ankle traction feature.

In another example, a pair of prior art traction devices 20, 20′ are illustrated with respect to FIGS. 1 and 2. Here, each of the prior art traction devices 20, 20′ include a sleeve 22 made from a compressive foam material generally formed into a structure designed to cup or otherwise at least partially enclose or harness most of a patient foot 24, an ankle 26 (hidden from view underneath the sleeve 22 in FIGS. 1 and 2), and at least a portion of a lower leg 28. The main difference in the structure of the prior art traction device 20 illustrated in FIG. 1 from the prior art traction device 20′ illustrated in FIG. 2 is that the prior art traction device 20′ includes an additional overcoating 30 that provides more structural support along the length of the lower leg 28 to just above the ankle 26, as illustrated, e.g., in FIG. 2.

Although, in each design, the compressive foam sleeve 22 is of a unibody construction being generally L-shaped, with an open anterior seam 32 running down the middle that permits selective insertion and/or removal of the foot 24, the ankle 26, and/or the lower leg 28 therein. Moreover, the sleeve 22 includes a pair of longitudinally extending supports 34, 34′ separately attached along the length of the sleeve 22 between where the sleeve 22 cups the lower leg 28 and the ankle 26. The pair of longitudinally extending supports 34, 34′ may help stabilize medial-to-lateral and lateral-to-medial movement thereof.

The prior art ankle traction devices 20, 20′ effectively operate by sliding the foot 24, the ankle 26, and the lower leg 28 of the patient through the open anterior seam 32 in the sleeve 22 into the position generally illustrated in FIGS. 1 and 2. From there, each of a set of straps 36 (three shown in FIGS. 1 and 2) respectively attached at one end to a first retaining ring 38 (e.g., stitched to the ankle traction device 20, 20′ along the length of the support 34) are pulled across the open anterior seam 32 and threaded through a corresponding second retaining ring 40 stitched to the ankle traction device 20, 20′ along the length of the support 34′ on an opposite side of the sleeve 22. Each of the straps 36 may then be drawn through the respective second retaining rings 40, back about themselves, and over the anterior seam 32. This causes the second set of retaining rings 40 to be drawn toward the first set of retaining rings 38 to help close the anterior seam 32 about the lower leg 28. At a minimum, each of the straps 36 obstruct movement of the lower leg 28 out from within the sleeve 22 by extending over the anterior seam 32, as illustrated in FIGS. 1 and 2.

Additionally, FIGS. 1 and 2 also illustrate that each of the longitudinally extending supports 34, 34′ extend beyond the bottom of the sleeve 22, and FIG. 2 further illustrates that each of the longitudinally extending supports 34, 34′ connect to a baseplate 42 positioned underneath the foot 24. The baseplate 42, e.g., may help provide support for the foot 24, and may include an anchor 44 coupled thereto that extends away from the foot 24 for use in placing the injured leg into traction, as is known in the art. Although, when in traction, the structure of the sleeve 22 is not able to provide adequate support in and around the entirety of the ankle 26 largely because the last of the straps 36 sits just above the foot 24 and the ankle 26 as illustrated in FIGS. 1 and 2. As such, the prior art traction devices 20, 20′ only provide a “harness”-type support that does not provide complete support in and around the entirety of the ankle 26 and otherwise does not include any sort of anterior support to help with load distribution. This is due, in large part, to the structure of the sleeve 22 being open along the anterior seam 32 to enable the foot 24, the ankle 26, and the lower leg 28 to slide into the sleeve 22 for select retainment therein.

There exists, therefore, a significant need in the art for a traction ankle wrap that fully and completely wraps in and around the entirety of the ankle to provide enhanced anterior support thereto, when used in connection with a traction splint. The present invention fulfills these needs and provides further related advantages.

### SUMMARY OF THE INVENTION

In one embodiment, the traction ankle wrap as disclosed herein is designed to provide anterior pressure to a patient foot during traction. In some embodiments, the traction ankle wrap may include a rear support from which an anterior strap outwardly extends from one side thereof and from which a closure strap outwardly extends from the opposite side thereof. Additionally, a base support may extend outwardly from the rear support between the anterior strap and the closure strap. These components, namely the rear support, the anterior strap, the closure strap, and the base support, may cooperate to form an enclosure having a size and shape to receive a patient foot and, when the traction ankle wrap is coupled thereto, apply anterior pressure to the patient foot during traction.

In some embodiments, the anterior strap may include a length that is at least partially extendable over the patient foot and the closure strap may include a length that is at least partially extendable over both the patient foot and the anterior strap. Moreover, the base support may extend outwardly from the rear support at an angle of approximately 90 degrees to provide additional structural stability. The traction ankle wrap may also be constructed from a stretchable material that flexibly conforms the rear support, the anterior strap, the closure strap, and the base support around the patient foot simultaneously and in a manner to apply anterior pressure to the patient foot when the foot is in traction.

To accommodate the heel, a cut-out may be formed from portions of the rear support and base support, thereby allowing at least partial insertion of the heel therein. The interior surface of the traction ankle wrap may include at least one instruction for proper attachment to the patient foot, such as the word “CALF” on the rear support or the word “HEEL” on the base support. In some embodiments, the closure strap may include an upper closure strap and a lower closure strap, wherein each of the anterior strap, the upper closure strap, and the lower closure strap may include an alphanumeric code visually identifying a sequence for attaching the traction ankle wrap to a patient foot. In some embodiments, the anterior strap may include the number “1”, the lower closure strap may include the number “2”, and the upper closure strap may include the number “3.”

Each of the upper and lower closure straps may include outwardly extending couplings (e.g., made from a nylon material), which further include sets of hooks or loops that are designed to selectively engage a corresponding set of hooks or loops on the exterior of the anterior strap. The upper and lower closure straps may then cooperate to retain the anterior strap thereunder in compression against the patient foot.

In some embodiments, an under-wrap may be positioned within the interior of the enclosure for added comfort or protection. The traction ankle wrap may also feature a unitary construction wherein the rear support, the closure strap, and the base support are cut or otherwise formed from a single piece of material. A pair of reinforcements may outwardly extend from the base support, with one reinforcement being coupled to the exterior of the rear support adjacent the anterior strap and the other reinforcement being coupled to the exterior of the rear support adjacent the closure strap. The traction ankle wrap may also have a heel strap having opposing ends respectively coupled to these reinforcements, and may downwardly extend out and around the exterior of the base support when attached thereto. Additionally, the heel strap may include a slide ring and a bolt snap slidably coupled thereto, wherein the slide ring is selectively attachable to a gauge and the bolt snap is selectively couplable to a scale cable system.

In another embodiment, the traction ankle wrap as disclosed herein may include a generally vertically positionable calf support and a generally horizontally positionable heel support projecting outward from a base of the calf support. An anterior foot support may flank a first side of the calf support and may have a width sufficient to fold back over and encompass at least a portion of the calf support. Additionally, a pair of closure straps may flank a second side of the calf support opposite the anterior foot support, each of which have a width sufficient to fold over and at least partially encompass the anterior foot support when folded over the calf support. Each of the calf support, the heel support, the anterior foot support, and the pair of closure straps may also include an alphanumeric character providing visual guidance for wrapping an enclosure around a patient foot to apply anterior pressure thereto when the patient foot is in traction.

In some embodiments, the traction ankle wrap may also be formed from a stretchable material that flexibly conforms the calf support, the heel support, the anterior foot support, and/or the pair of closure straps to the patient foot simultaneously while applying the anterior pressure. A contiguous cut-out may be formed from portions of the calf support and the heel support to accommodate insertion of a patient heel therein. The alphanumeric characters may include the word “CALF” on the calf support, the word “HEEL” on the heel support, the number “1” on the anterior foot support, the number “2” on one closure strap (e.g., a lower closure strap), and the number “3” on the other closure strap (e.g., an upper closure strap), thereby providing a clear sequence of instructions for attachment.

Each closure strap may also include an outwardly extending fastener having a set of hooks or loops selectively removably engageable with a corresponding set of hooks or loops on the anterior foot support. These fasteners may be made of nylon material, and the closure straps may cooperate to retain the anterior foot support underneath in compression against the patient foot. In some embodiments, the traction ankle wrap may be of a unitary construction such that the calf support, the heel support, the anterior foot support, the closure straps, and a pair of reinforcements outwardly extending from the heel support and attached to an exterior surface of the calf support are formed from a single piece of material.

Additionally, in some embodiments, a heel strap may have opposing ends respectively coupled to each of the pair of reinforcements and may downwardly extend out and around the exterior of the heel support. Furthermore, the heel strap may include a slide ring and a bolt snap slidably coupled thereto, wherein the slide ring is selectively attachable to a gauge and the bolt snap is selectively couplable to a scale cable system.

In another alternative embodiment, a process for applying a traction ankle wrap to a patient foot may include inserting the patient foot into a four-sided enclosure generally defined by a rear support, an anterior strap, a closure strap, and a base support. Here, the patient foot may land on the base support and be positioned up against an interior surface of the rear support such that the heel resides within a cut-out formed from portions of the rear support and the base support. Once positioned, the anterior strap may extend over an anterior portion of the patient foot toward the closure strap and be tucked thereunder. In some embodiments where the closure strap includes an upper closure strap and a lower closure strap, the anterior strap may tuck underneath both of the upper closure strap and the lower closure strap.

Next, one or both of the closure straps, depending on the embodiment, may be tightened about the anterior strap, thereby placing additional anterior pressure on the patient foot. In some embodiments, one or both of the closure straps may stretch over the exterior surface of the anterior strap in compression-fit relation and attach thereto by a fastener. In some embodiments, the anterior strap may bear the number “1”, the lower closure strap may bear the number “2”, and the upper closure strap may bear the number “3”, wherein these alphanumeric indicators identify the sequence for wrapping the traction ankle wrap around the patient foot.

The process may further include coupling a slide ring to a gauge and a bolt strap to a scale cable system, wherein traction on the traction ankle wrap may be exerted via the scale cable system. The slide ring and the bolt strap may be positioned along a heel strap that downwardly extends out and around the exterior of the base support. Real-time gauging of traction force may be performed using the scale cable system, and in some embodiments, the anterior pressure applied to the patient foot may include between 10 and 15 pounds of force.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the traction ankle wrap. In such drawings:

FIG. 1 is a left-side perspective view of a prior art traction device having a foam sleeve with an open anterior seam;

FIG. 2 is a right-side perspective view of a prior art traction device similar to that illustrated in FIG. 1, further illustrating that the foam sleeve includes a reinforced overcoating;

FIG. 3 is a front perspective view of a traction ankle wrap as disclosed herein;

FIG. 4 is a top perspective view of the traction ankle wrap of FIG. 3;

FIG. 5 is a front perspective environmental view illustrating inserting a patient foot into the traction ankle wrap of FIGS. 3 and 4;

FIG. 6 is a front perspective environmental view illustrating wrapping an anterior strap about the patient foot;

FIG. 7 is a front perspective environmental view illustrating the front anterior strap secured about the patient foot;

FIG. 8 is a front perspective environmental view illustrating wrapping a lower closure strap about the anterior strap;

FIG. 9 is a front perspective environmental view illustrating wrapping an upper closure strap about the anterior strap;

FIG. 10 is a side perspective environmental view illustrating the lower closure strap and the upper closure strap secured over the anterior strap, wherein the traction ankle wrap is attached to a patient foot, ankle, and leg;

FIG. 11 is a bottom perspective environmental view illustrating moving a slide ring underneath the traction ankle wrap attached to the patient foot;

FIG. 12 is a bottom perspective environmental view similar to FIG. 11, further illustrating aligning the slide ring with a bolt snap prior to attachment to a traction splint;

FIG. 13 is a bottom perspective environmental view illustrating connecting the slide ring to a U-shaped clip at a distal end of a traction gauge;

FIG. 14 is a side perspective environmental view, further illustrating the patient foot with the traction ankle wrap thereon at rest while attached to the traction gauge;

FIG. 15 is an enlarged side perspective environmental view taken about the circle 15 in FIG. 14, further illustrating the slide ring attached to the traction gauge by the U-shaped clip;

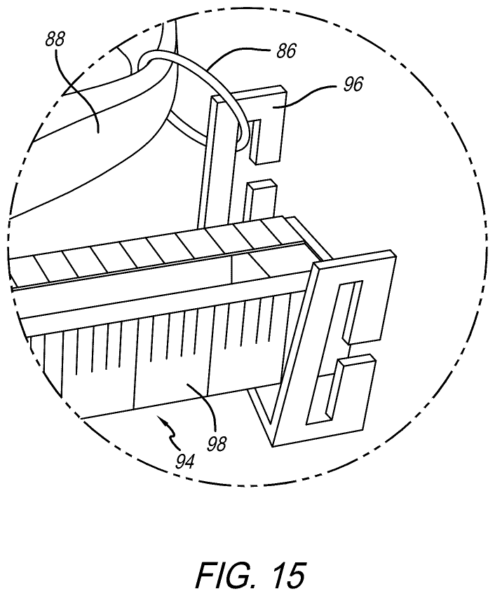

FIG. 16 is a side perspective environmental view illustrating the bolt snap connected to a scale cable system;

FIG. 17 is an enlarged side perspective environmental view taken about the circle 17 in FIG. 16, further illustrating the bolt snap connected to the scale cable system;

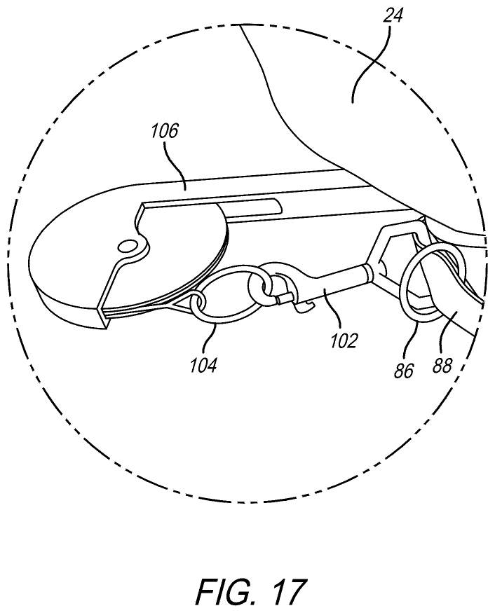

FIG. 18 is a top plan view of the traction ankle wrap in a cutting layout; and

FIG. 19 is a side perspective view of the traction ankle wrap, further illustrating a side support attached to a vertical section of the traction ankle wrap immediately behind each of the upper closure strap and the lower closure strap.

### DETAILED DESCRIPTION OF THE PREFERRED EMBODIMENTS

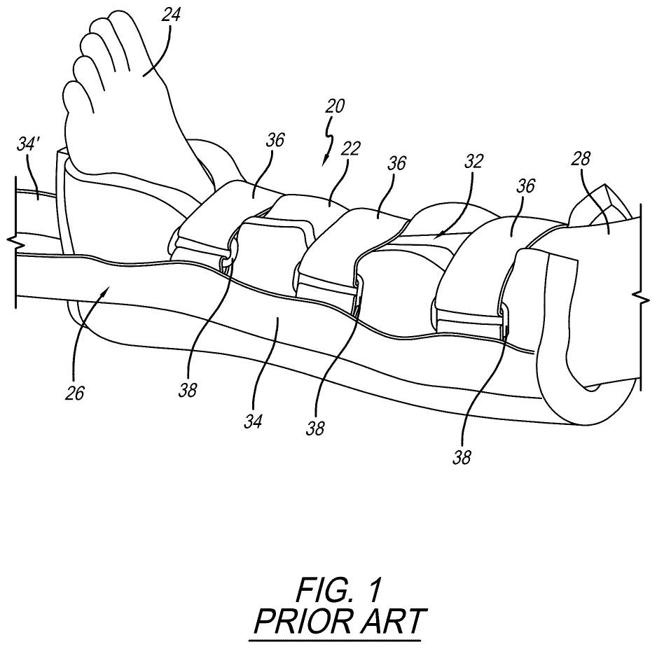

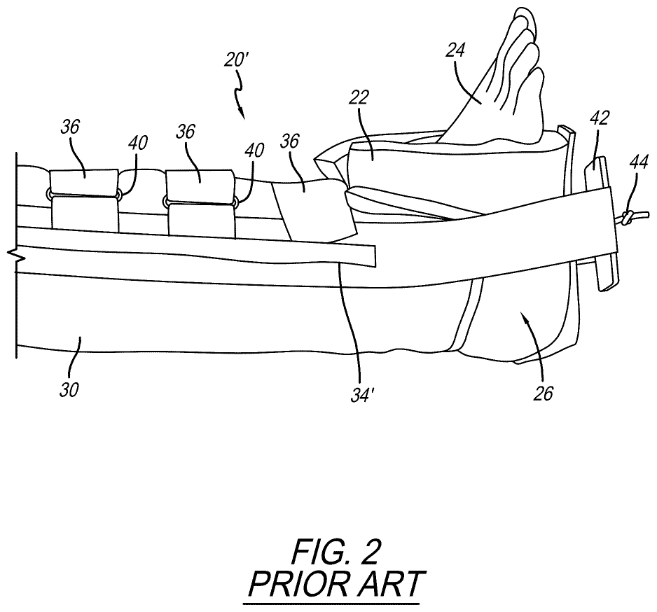

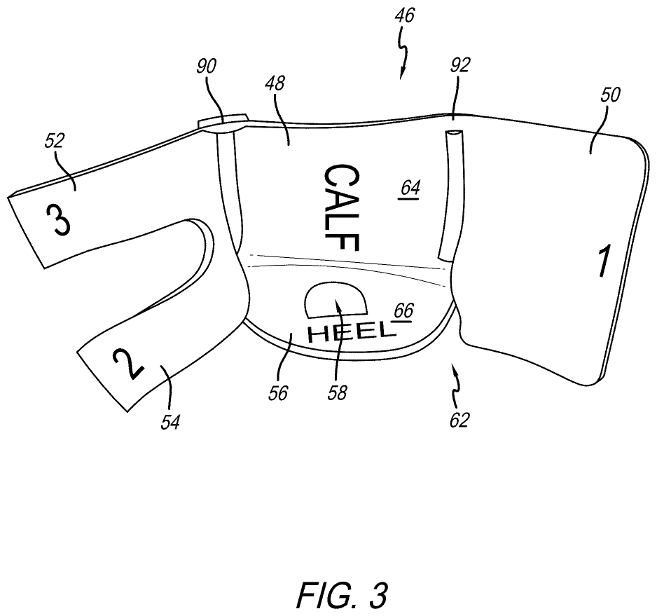

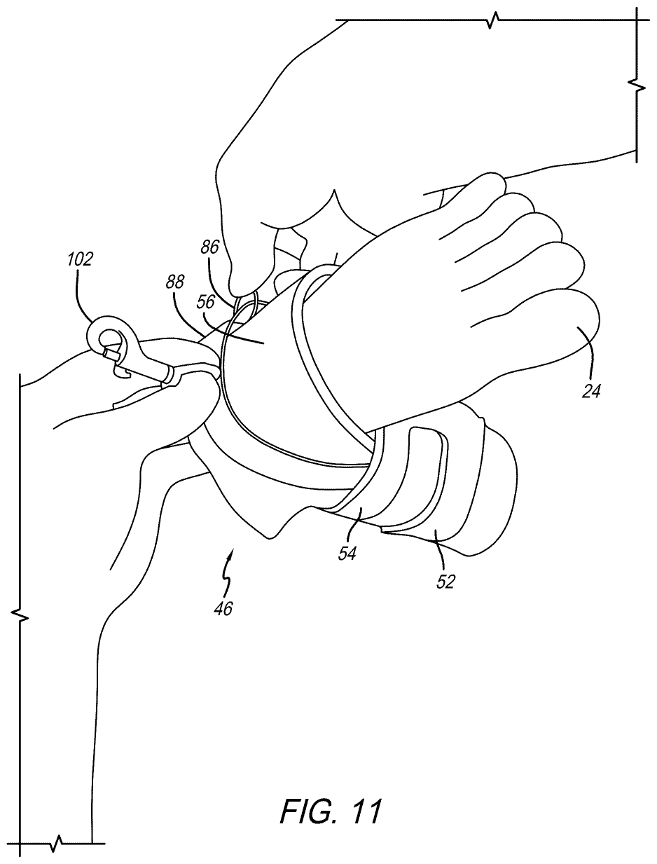

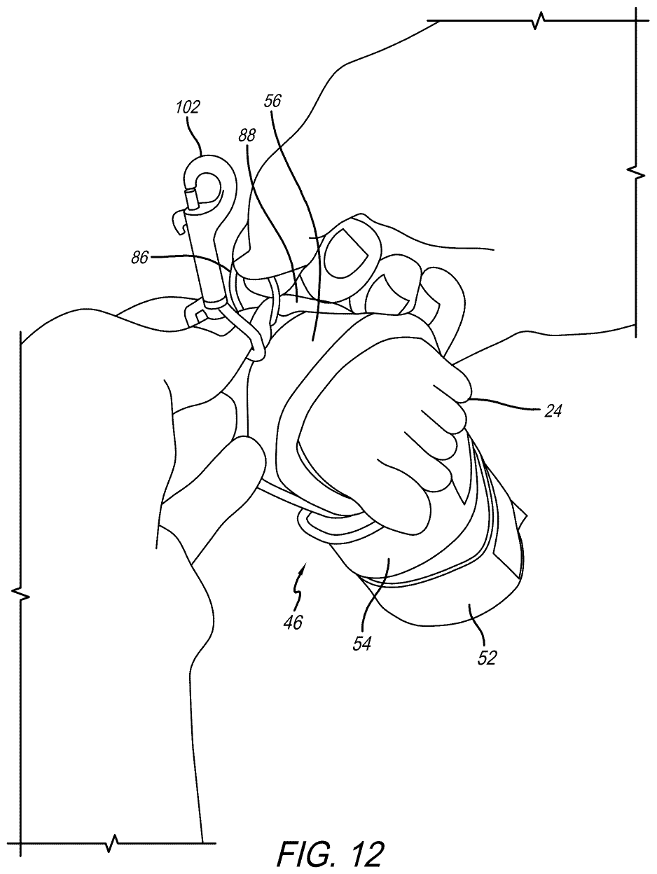

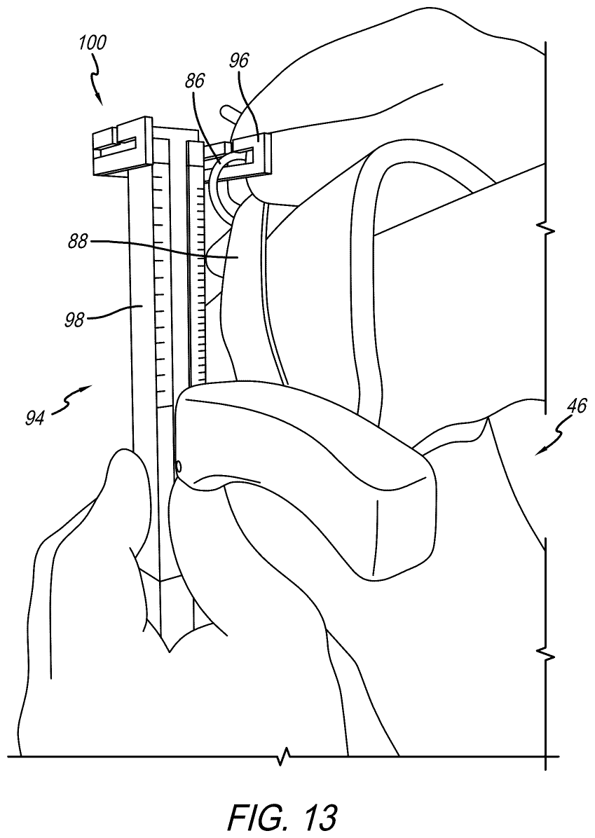

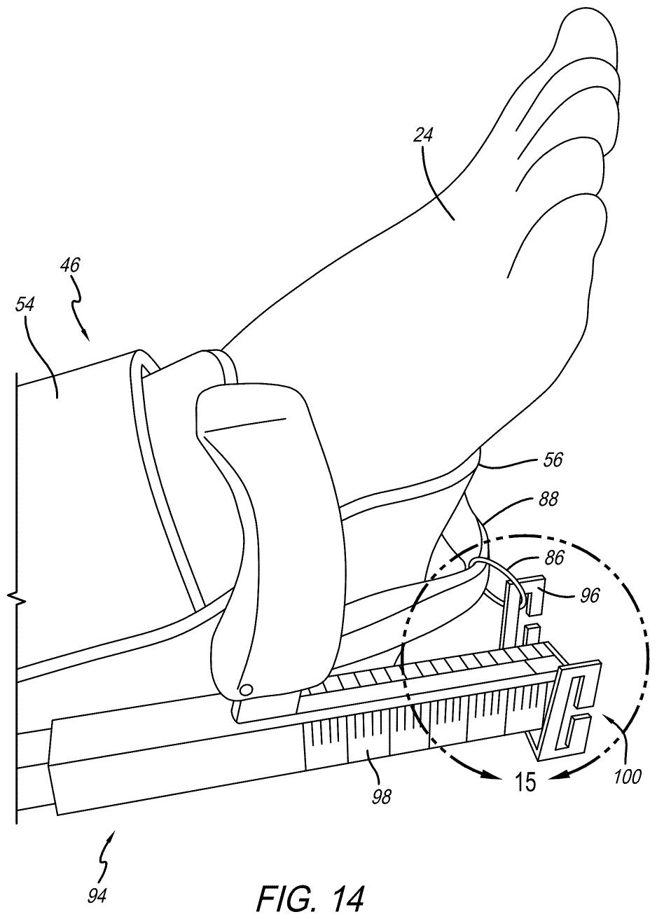

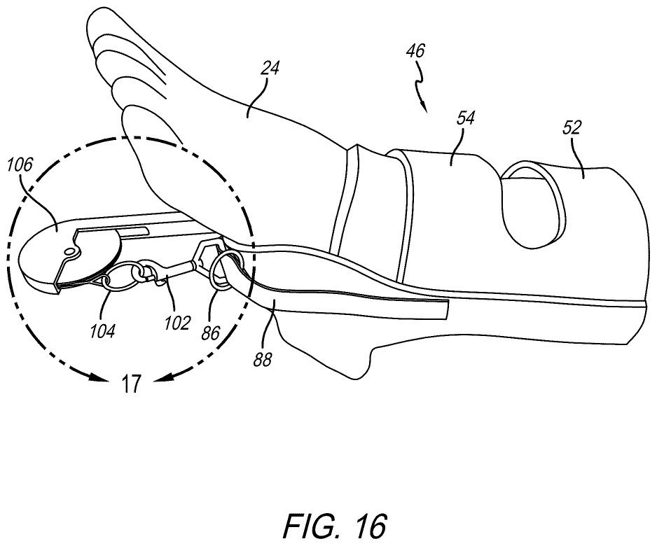

As shown in the exemplary drawings for purposes of illustration, the present disclosure for a traction ankle wrap is generally referred to by reference numeral 46 in FIGS. 3-14, 16, and 18-19. The structure of the traction ankle wrap 46 disclosed herein, as will be shown and described in more detail below, provides better conformance to the anatomy of the foot 24, the ankle 26, and at least a portion of the lower leg 28 relative to comparable devices known in the art, including, e.g., the sleeve 22 used in connection with the prior art ankle traction devices 20, 20′ discussed above with respect to FIGS. 1 and 2. For example, in one embodiment, the traction ankle wrap 46 disclosed herein may be made from a stretchable material (e.g., a polychloroprene material such as neoprene) and formed into a “boot”-like structure having a series of closures intuitively designed to quickly and easily snugly wrap the traction ankle wrap 46 around the ankle 26 of a patient in a manner that places relatively even pressure in and around the entire ankle 26 while simultaneously providing enhanced anterior support thereto (e.g., during traction). In one embodiment, the traction ankle wrap 46 is able to apply a relatively even pressure between 10 and 15 pounds of force to the lower extremity of a patient during traction. Moreover, such a stretchable material may include additional padding to provide enhanced comfort and protection for the skin against the rigid parts of the splint.

The ease of use to achieve the desired traction with the traction ankle wrap 46 may be particularly beneficial in the “field” during an emergency when a patient with an injury may need to be placed into traction immediately to stabilize a femur fracture for purposes of quickly transporting the patient to a hospital. As such, in one embodiment, the traction ankle wrap 46 disclosed herein may generally be compact, lightweight, and easily storable in a medical vehicle, such as an ambulance, for transportation to an emergency. Moreover, the traction ankle wrap 46 disclosed herein may also be adapted for use as a component part of a field traction device, such as the SagerX Field Splint marketed and sold by SunMedica, Inc. of 1661 Zachi Way, Redding, California 96003.

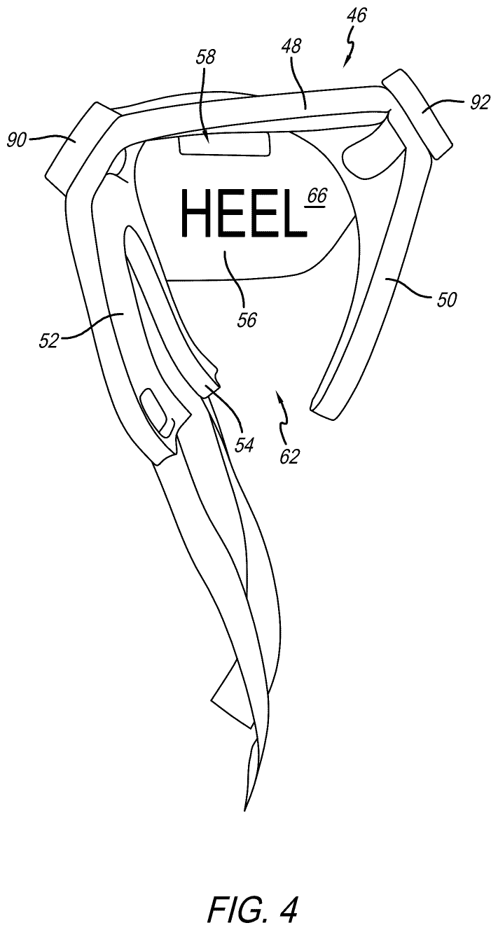

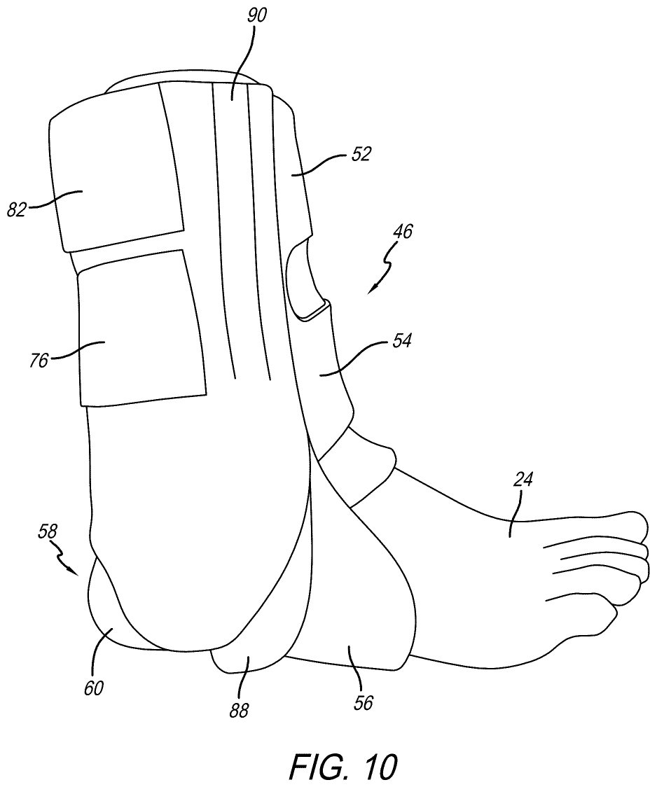

More specifically, in one embodiment, FIGS. 3 and 4 illustrate that the traction ankle wrap 46 includes a rear support 48 having an anterior strap 50 outwardly projecting from one side thereof and an upper closure strap 52 and a lower closure strap 54 outwardly projecting therefrom from another side of the rear support 48 opposite the anterior strap 50. FIGS. 3 and 4 also illustrate that the traction ankle wrap 46 may include a base support 56 outwardly projecting from the rear support 48 at approximately 90 degrees relative thereto, with a cut-out 58 being formed from a portion of the rear support 48 in the horizontal plane and formed from a portion of the base support 56 in a vertical plane. The cut-out 58 provides a location where a heel 60 of a patient, e.g., as best illustrated in FIG. 10, may slide into to more snugly attach the traction ankle wrap 46 to the patient give the curvature of the bottom of the foot 24. Together, the rear support 48, the anterior strap 50, the upper closure strap 52, the lower closure strap 54, and the base support 56 cooperate to form an enclosure 62 that generally receives the patient foot and provides support in and around the entirety of the ankle 26, including anteriorly.

In one embodiment, each of the rear support 48, the anterior strap 50, the upper closure strap 52, the lower closure strap 54, and/or the base support 56 may include one or more markings thereon to provide guidance how to position and attach the traction ankle wrap 46 to a patient. Such markings may be particularly beneficial for use in an emergency as the basic instructions for locating and attaching the traction ankle wrap 46 to an injured patient are basically written on the surfaces of the traction ankle wrap 46 itself.

For instance, as illustrated in FIG. 3, the rear support 48 includes the word “CALF” written along the height of an interior surface 64 thereof to provide an immediate visual indicator where to locate the patient calf. Similarly, FIGS. 3 and 4 illustrate that the base support 56 may include the word “HEEL” on its interior surface 66 to provide an immediate visual indicator where to locate the patient heel. Although, of course, other designations may be written to each of the rear support 48 and/or the base support 56 to provide visual indication regarding where to locate the patient foot and/or heel. For example, the words “CALF” and “HEEL” may be replaced by other words, phrases, signs, or indicators, such as drawings illustrating where to locate certain anatomical features to appropriately attach the traction ankle wrap 46 to the patient.

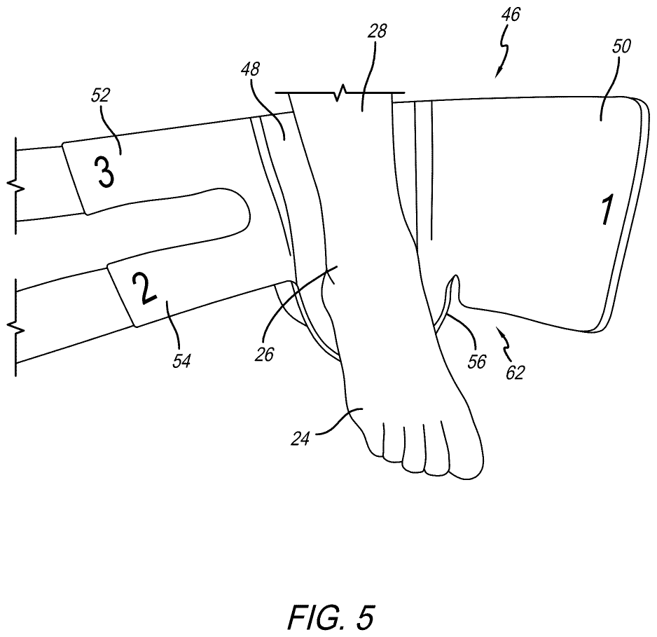

As such, these visual indicators provide an easily recognizable set of instructions where to locate the calf of the patient (i.e., along the interior surface 64 of the rear support 48 having the word CALF thereon) and where to locate the heel of the patient (i.e., on the interior surface 66 of the base support 56 with the word HEEL thereon). As such, emergency personnel can immediately orient the traction ankle wrap 46 so that the foot 24, the ankle 26, and the leg 28 are initially positioned relative to the traction ankle wrap 46 as illustrated in FIG. 5, i.e., with the calf of the patient pressed against the “CALF” marking (or other visual designation, as desired) on the interior surface 64 of the rear support 48 and the heel of the patient landed on the “HEEL” marking (or other visual designation, as desired) on the interior surface 66 of the base support 56. When in this initial position, at least a portion of the heel 60 may slide into engagement and stick out from the cut-out 58, as mentioned above and illustrated in FIG. 10.

Additionally, each of the straps 50, 52, 54 may include an alphanumeric code designating the sequence for attaching each of the straps 50, 52, 54 to the patient. For instance, as illustrated in FIGS. 3 and 5-9, the anterior strap 50 may include the number “1”, the upper closure strap 52 may include the number “3”, and the lower closure strap 54 may include the number “2”. As such, the sequence for attaching the straps 50, 52, 54 to the patient would start with the anterior strap 50 (designated by the number “1”), followed by the lower closure strap 54 (designated by the number “2”) and finally ending with the upper closure strap 52 (designated by the number “3”). Although, of course, other designations may be written to each of the straps 50, 52, 54 to provide visual indication regarding the sequence each of the straps 50, 52, 54 should attached to the patient. For example, the numbers may be replaced by words, phrases, or signs or indicators such as arrows providing guidance as to the order for attaching each of the straps 50, 52, 54 around the foot 24, the ankle 26, and the leg 28 of the patient, for purposes of securing the traction ankle wrap 46 thereto more quickly and efficiently, especially in an emergency.

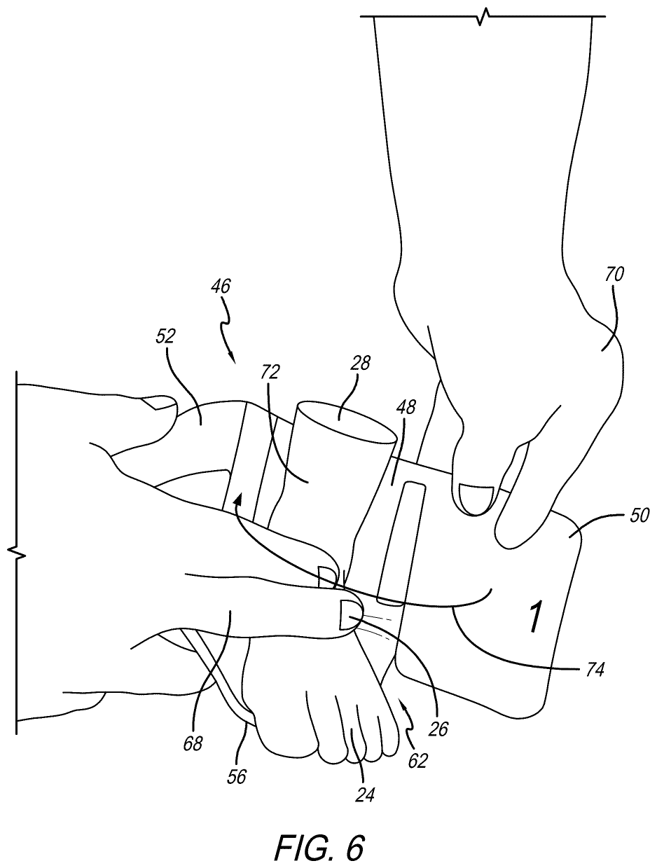

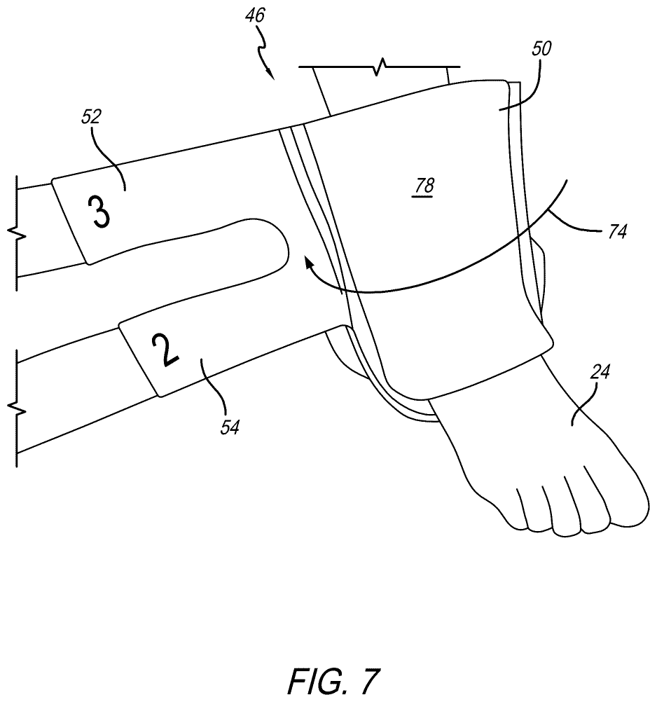

Once the foot 24 of the patient is located within the area designated for the “heel” on the base support 56 and the area designated for the “calf” on the rear support 48, e.g., as illustrated in FIG. 5, one or both of the upper closure strap 52 and the lower closure strap 54 may be held with a first hand 68 so that a second hand 70 may grasp and pull/stretch the anterior strap 50 having the number “1” thereon across a shin 72 of the ankle 26, such as along a directional arrow 74 (FIGS. 6 and 7). Once across the shin 72, the anterior strap 50 should be tucked in underneath each of the upper closure strap 52 and the lower closure strap 54, e.g., in the position illustrated in FIG. 7. At the same time, the first hand 68 may simultaneously apply some pressure to the shin 72 to push the lower leg 28 back into engagement with the interior surface 64 of the rear support 48 so that the anterior strap 50 snuggly wraps around the posterior of the lower leg 28, the posterior of the ankle 26, and around the shin 72. The flexibility of the neoprene material of the traction ankle wrap 46 enables the rear support 48 and the anterior strap 50 to conform to the structure of the foot 24, the ankle 26, and the leg 28, including effectively applying pressure anteriorly along the foot 24 and the ankle 26 when attached thereto.

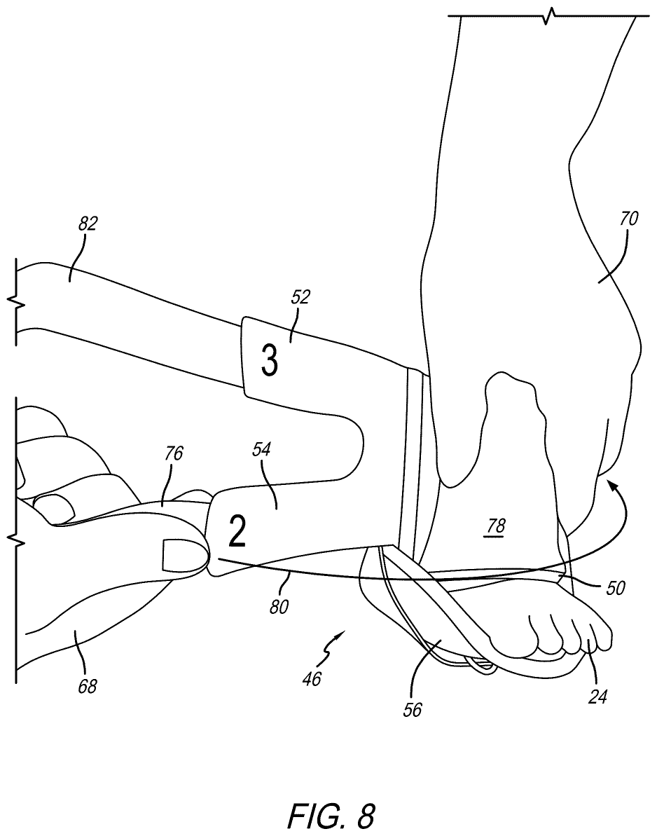

Once in the position illustrated in FIG. 7, the anterior strap 50 may be held to the patient as illustrated in FIG. 8 (e.g., with the second hand 70), including maintaining the anterior strap 50 tucked up underneath each of the upper closure strap 52 and/or the lower closure strap 54 in preparation for wrapping the lower closure strap 54 back across an exterior surface 78 of the anterior strap 50. In this respect, the next step is to grasp a coupling 76 (e.g., a Velcro strip) extending out from the lower closure strap 54 (FIG. 8), and then pull the lower closure strap 54 over the exterior surface 78 of the anterior strap 50, such as along a directional arrow 80 illustrated in FIG. 8. Again, the number “2” associated with the lower closure strap 54 provides an immediate visual indication this is the next of the straps 50, 52, 54 to attach the traction ankle wrap 46 to the patient. The coupling 76 may include a series of hooks or loops that selectively attach to a series of hooks and/or loops formed from the exterior surface 78 of the anterior strap 50. This way, the lower closure strap 54 may firmly and securely attach to the anterior strap 50 by stretching the lower closure strap 54 to whatever length and tightness is deemed appropriate to provide additional anterior support across the foot 24 and/or the ankle 26.

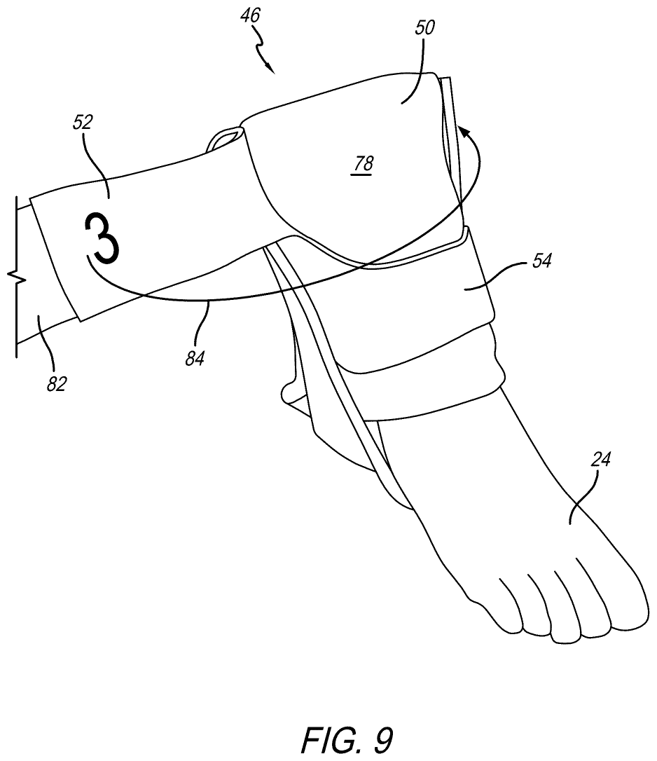

Once the ankle traction wrap 46 is in the position illustrated in FIG. 9, and while continuing to hold the anterior strap 50 in the position illustrated in FIG. 8 (e.g., now with the lower closure strap 54 secured and positioned underneath the second hand 70), the upper closure strap 52 may be pulled back across the exterior surface 78 of the anterior strap 50 to the position illustrated in FIG. 10. Again, the number “3” associated with the upper closure strap 52 provides an immediate visual indication this is the next (and last) of the straps 50, 52, 54 for attaching the traction ankle wrap 46 to the patient. In this respect, the first hand 68 (not illustrated in FIG. 9) grasps a coupling 82 (e.g., another Velcro strip) extending out from the upper closure strap 52, to pull the upper closure strap 52 over the exterior surface 78 of the anterior strap 50, such as along a directional arrow 84 illustrated in FIG. 9. Similarly, the coupling 82 may include a series of hooks or loops that selectively attach to a series of hooks and/or loops formed from the exterior surface 78 of the anterior strap 50. This way, like the lower closure strap 54, the upper closure strap 52 may firmly and securely attach to the anterior strap 50 by stretching the upper closure strap 52 to whatever length and tightness is deemed appropriate to provide additional anterior support to the ankle 26 and to the lower leg 28.

As such, each of the upper closure strap 52 and the lower closure strap 54 snugly hold the anterior strap 50 thereunder and in compression with the foot 24 and/or the ankle 26, including that the traction ankle wrap 46 includes material positioned to place anterior pressure and support thereto. Such wrapping of the support 48, the anterior strap 50, the upper closure strap 52, the lower closure strap 54, and the base support 56 around the foot 24, the ankle 26, and a portion of the lower leg 28 provides better load distribution over the entirety thereof as a result of being fully compressively wrapped therearound.

While the couplings 76, 82 discussed above include hook/loop attachments such as Velcro, and may be made from a nylon material, the couplings 76, 82 may be another fastening device known in the art, such as an adhesive strap, button mechanism, or another mechanical attachment designed to retain the straps 50, 52, 54 about the patient. As such, in each of these embodiments, the couplings 76, 82 should be positioned away from the patient so as to avoid touching the skin because the stretchable neoprene material is much softer and better conforms to the contours of the foot 24, the ankle 26, and/or the lower leg 28. This not only ensures better comfort, but a higher degree of surface-to-surface engagement in and around the foot 24, the ankle 26, and the lower leg 28 to ensure conformed attachment of the traction ankle wrap 46 thereto, and more even support, including anteriorly. As such, this combination of the couplings 76, 82 and the stretchable neoprene material from which each of the rear support 48, the anterior support 50, the upper closure strap 52, the lower closure strap 54, and the base support 56 are made results in a conforming and well padded “boot” that can be used as a source of traction.

Specifically, the stretchable neoprene material facilitates flush engagement to enable the relatively soft and flexible material forming each of the rear support 48, the anterior strap 50, the upper closure strap 52, the lower closure strap 54, and the base support 56 to form in and around the contours of the foot 24, the ankle 26, and the leg 28 to provide the desired stabilization to patients that vary in size, after placing the lower extremity into traction. An additional cotton under-wrap (not shown) may be positioned between the rear support 48, the anterior strap 50, the upper closure strap 52, the lower closure strap 54, and/or the base support 56 and the skin of the foot 24, the ankle 26, and/or the leg 28 to provide additional comfort and to reduce slippage of the traction ankle wrap 46 after being attached to the patient.

Ideally, shoes and/or socks worn by the patient are removed before application of the traction ankle wrap 46 so that each of the rear support 48, the anterior strap 50, the upper closure strap 52, the lower closure strap 54, and the base support 56 can wrap flush against the foot 24, the ankle 26, and/or to at least a portion of the lower leg 28, as disclosed above. Although, the traction ankle wrap 46 may also be used over socks and/or shoes because the traction ankle wrap 46 is stretchable. In this respect, the stretchable neoprene material helps facilitate attachment of the traction ankle wrap 46 to patients that vary in size, including those that may be wearing socks and/or shoes. The traction ankle wrap 46 may be made in one size fits all construction, or may come in various sizes to accommodate patients that vary in size (e.g., specific for children, adolescents, and/or adults). While a shoe and/or a sock may prevent tight conformity to the foot 24, the ankle 26, and/or the lower leg 28, the traction ankle wrap 46 may still be able to secure in and around the foot 24, the ankle 26, and the leg 28 over a sock or shoe for purposes of use adequate to place the limb in traction with a traction splint, e.g., for purposes of emergency transportation.

After wrapping the traction ankle wrap 46 to the foot 24, the ankle 26, and about a portion of the lower leg 28, the traction ankle wrap 46 may be easily attached to a femoral traction splint, such as the Sager® field splint marketed and sold by SunMedica, Inc. of 1661 Zachi Way, Redding, California 96003, as mentioned above. In this respect, a slide ring 86 attached to a heel strap 88 downwardly extending from a pair of side supports 90, 92 may attach to a gauge 94 (e.g., the Sager® D-S304-100) for purposes of measuring traction created by the Sager® field splint. Here, the slide ring 86 may engage a generally U-shaped clip 96 extending from a scale 98 at a distal end 100 thereof. Moreover, a bolt snap 102 also slidably attached to the heel strap 88 may couple to a key ring 104 attached to a scale cable system 106. The scale cable system 106 is able to apply traction using the traction ankle wrap 46 couple thereto, and the gauge 94 may provide real time measurements regarding the amount of traction applied with the traction ankle wrap 46 by way of the scale 98.

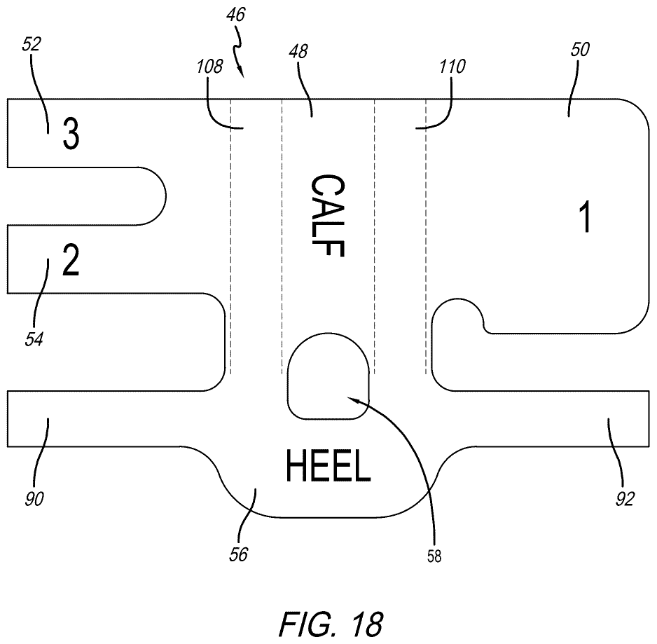

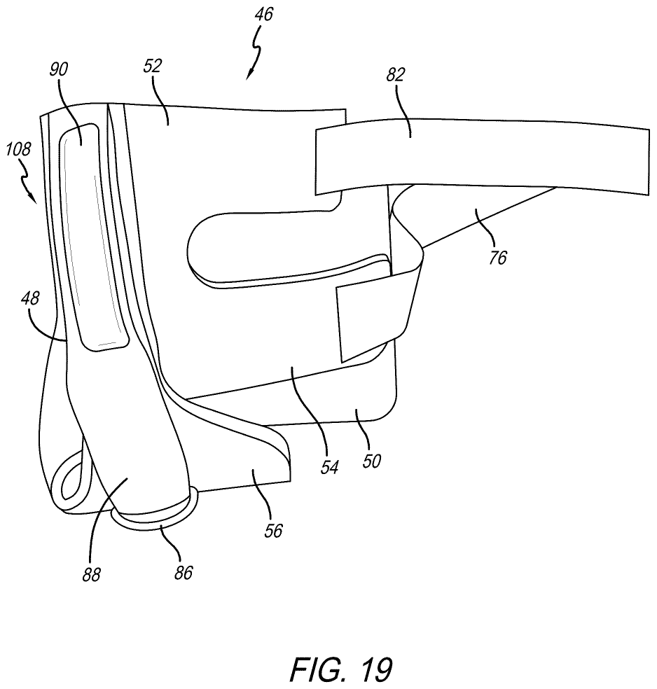

Lastly, the traction ankle wrap 46 may be made from a single piece of neoprene material, such as being cut according to the layout illustrated in FIG. 18. Once cut to the pattern illustrated in FIG. 18, each of the side supports 90, 92 fold back behind and attach to the material behind a pair of vertical sections 108, 110 positioned between the rear support 48 and the anterior strap 50 and each of the upper closure strap 52 and the lower closure strap 54. In this respect, the side supports 90, 92 are able to attach along medial and lateral sides of the traction ankle wrap 46, and in a similar position as the longitudinally extending supports 34, 34′. Similarly, the side supports 90, 92 may help stabilize medial-to-lateral and lateral-to-medial movement thereof. More specifically, FIG. 19 illustrates one embodiment wherein the side support 90 is coupled to the rear vertical section 108, with the heel strap 88 stitched thereover. FIG. 19 also illustrates that each of the couplings 76, 82 may be stitched to ends of the upper closure strap 52 and the lower closure strap 54, respectively.

Although several embodiments have been described in detail for purposes of illustration, various modifications may be made without departing from the scope and spirit of the invention. Accordingly, the invention is not to be limited, except as by the appended claims.

What is claimed is:

1. A traction ankle wrap, comprising:
a rear support having an anterior strap outwardly extending from one side thereof;
a closure strap outwardly extending from another side of the rear support opposite the anterior strap; and
a base support outwardly extending from the rear support in between the anterior strap and the closure strap;
wherein each of the rear support, the anterior strap, the closure strap, and the base support cooperate to form an enclosure having a size and shape for receiving a patient foot therein and, when coupled thereto, applying an anterior pressure on the patient foot when in traction.

2. The traction ankle wrap of claim 1, wherein the anterior strap comprises a length at least partially extendable over the patient foot and the closure strap comprises a length at least partially extendable over each of the patient foot and the anterior strap.

3. The traction ankle wrap of claim 1, wherein the base support extends outwardly from the rear support at an angle of approximately 90 degrees relative thereto.

4. The traction ankle wrap of claim 1, wherein the traction ankle wrap comprises a stretchable material flexibly conforming the rear support, the anterior strap, the closure strap, and the base support around the patient foot simultaneously while applying the anterior pressure to the patient foot.

5. The traction ankle wrap of claim 1, including a cut-out formed from a portion of the rear support and a portion of the base support for accommodating at least partial insertion of a heel of the patient foot.

6. The traction ankle wrap of claim 1, wherein an interior surface of the traction ankle wrap includes at least one instruction for attaching the traction ankle wrap to the patient foot.

7. The traction ankle wrap of claim 6, wherein the instruction comprises a word “CALF” on the rear support or a word “HEEL” on the base support.

8. The traction ankle wrap of claim 1, wherein the closure strap comprises an upper closure strap and a lower closure strap, and each of the anterior strap, the upper closure strap, and the lower closure strap include an alphanumeric code visually identifying a sequence for attaching the traction ankle wrap to the patient foot.

9. The traction ankle wrap of claim 8, wherein the alphanumeric code of the anterior strap comprises a number “1”, the alphanumeric code of the lower closure strap comprises a number “2”, and the alphanumeric code of the upper closure strap comprises a number “3”.

10. The traction ankle wrap of claim 8, wherein each of the upper closure strap and the lower closure strap include an outwardly extending coupling.

11. The traction ankle wrap of claim 10, wherein each of the outwardly extending couplings include a set of hooks or a set of loops selectively removably engageable with a set of hooks or a set of loops on an exterior of the anterior strap.

12. The traction ankle wrap of claim 10, wherein each coupling comprises a nylon material.

13. The traction ankle wrap of claim 8, wherein each of the upper closure strap and the lower closure strap cooperate to retain the anterior strap thereunder in compression with the patient foot.

14. The traction ankle wrap of claim 1, including an under-wrap positioned within an interior of the enclosure.

15. The traction ankle wrap of claim 1, wherein the traction ankle wrap comprises a unitary construction where each of the rear support, the closure strap, and the base support are cut from a single piece of material.

16. The traction ankle wrap of claim 15, including a pair of reinforcements outwardly extending from the base support, wherein one of the pair of reinforcements is coupled to an exterior of the rear support adjacent the anterior strap and the other of the pair of reinforcements is coupled to an exterior of the rear support adjacent the closure strap.

17. The traction ankle wrap of claim 16, including a heel strap having opposing ends respectively coupled to each of the pair of reinforcements, the heel strap downwardly extending out and around an exterior of the base support.

18. The traction ankle wrap of claim 17, including a slide ring and a bolt snap slidably coupled with the heel strap, the slide ring being selectively attachable to a gauge and the bolt strap being selectively couplable to a scale cable system.

19. A traction ankle wrap, comprising:
a generally vertically positionable calf support;
a generally horizontally positionable heel support projecting outward from a base of the calf support;
an anterior foot support flanking a first side of the calf support and having a width sufficient to fold back over and encompass at least a portion of the calf support; and
a pair of closure straps flanking a second side of the calf support opposite the first side, wherein each of the pair of closure straps have a width sufficient to fold over and at least partially encompass the anterior foot support folded over the calf support;
wherein each of the calf support, the heel support, the anterior foot support, and the pair of closure straps includes an alphanumeric character providing visual guidance for wrapping an enclosure around a patient foot and applying anterior pressure thereto when the patient foot is in traction.

20. The traction ankle wrap of claim 19, wherein the traction ankle wrap comprises a stretchable material flexibly conforming the calf support, the heel support, the anterior foot support, and the pair of closure straps to the patient foot simultaneously while applying the anterior pressure thereto.

21. The traction ankle wrap of claim 19, including a contiguous cut-out formed from a portion of the calf support and a portion of the heel support.

22. The traction ankle wrap of claim 19, wherein the alphanumeric character of the calf support comprises a word “CALF”, the alphanumeric character of the heel support comprises a word “HEEL”, the alphanumeric character of the anterior foot support comprises a number “1”, the alphanumeric character of one of the pair of closure straps comprises a number “2”, and the alphanumeric character of the other of the pair of closure straps comprises a number “3”.

23. The traction ankle wrap of claim 19, wherein each of the pair of closure straps include an outwardly extending fastener having a set of hooks or a set of loops selectively removably engageable with a set of hooks or a set of loops of the anterior foot support.

24. The traction ankle wrap of claim 23, wherein each fastener comprises a nylon material and each of the pair of closure straps cooperate to retain the anterior foot support thereunder in compression with the patient foot.

25. The traction ankle wrap of claim 19, wherein the traction ankle wrap comprises a unitary construction such that each of the calf support, the heel support, the anterior foot support, the pair of closure straps, and a pair of reinforcements outwardly extending from the heel support and attached to an exterior surface of the calf support are formed from a single piece of material.

26. The traction ankle wrap of claim 25, including a heel strap having opposing ends respectively coupled to each of the pair of reinforcements and downwardly extending out and around an exterior of the heel support, wherein a slide ring and a bolt snap slidably couple with the heel strap and respectively selectively attach to a gauge and a scale cable system.

27. A process for applying a traction ankle wrap to a patient foot, comprising the steps of:
inserting the patient foot into a four-sided enclosure generally defined by a rear support, an anterior strap, a closure strap, and a base support;
extending the anterior strap over an anterior portion of the patient foot toward the closure strap; and
stretching the closure strap over an exterior surface of the anterior strap in compression-fit relation therewith such that each of the rear support, the anterior strap, the closure strap, and the base support hug the patient foot and cooperate to apply an anterior pressure thereon when the patient foot is in traction.

28. The process of claim 27, wherein the inserting step includes the step of landing the patient foot on the base support and positioning the patient foot against an interior surface of the rear support such that a heel of the patient foot resides within a cut-out formed from a portion of the rear support and a portion of the base support.

29. The process of claim 27, wherein the extending step includes the step of tucking the anterior strap underneath the closure strap.

30. The process of claim 29, wherein the closure strap includes an upper closure strap and a lower closure strap, and the tucking step includes the step of tucking the anterior strap underneath both the upper closure strap and the lower closure strap.

31. The process of claim 30, including the step of tightening the upper closure strap and the lower closure strap about the anterior strap, thereby placing additional anterior pressure on the patient foot.

32. The process of claim 27, wherein the stretching step includes the step of attaching the closure strap and a second closure strap to the exterior surface of the anterior strap by a fastener.

33. The process of claim 32, wherein the closure strap comprises a lower closure strap having a number “2” thereon, the second closure strap comprises an upper closure strap having a number “3” thereon, and the anterior strap has a number “1” thereon, wherein each of the number 1, the number 2, and the number 3 identify a sequence that each of the anterior strap, the lower closure strap, and the upper closure strap wrap about the patient foot.

34. The process of claim 27, including the step of coupling a slide ring to a gauge and a bolt strap to a scale cable system, and exerting traction on the traction ankle wrap with the scale cable system.

35. The process of claim 34, including the step of positioning the slide ring and the bolt strap along a heel strap downwardly extending out and around an exterior of the base support.

36. The process of claim 35, including the step of gauging a traction force in real-time with the scale cable system.

37. The process of claim 37, wherein the anterior pressure comprises between 10 and 15 pounds of force.
